(19) United States
(12) Patent Application Publication
(10) Pub. No.: US 2026/0159554 A1
(43) Pub. Date: Jun. 11, 2026

(54) EUKARYOTIC ALGAE COMPOSITIONS AND METHODS THEREOF

(71) Applicant: COLORADO STATE UNIVERSITY RESEARCH FOUNDATION, Fort Collins, CO (US)

(72) Inventors: Graham PEERS, Fort Collins, CO (US); Tessema KASSAW, Fort Collins, CO (US)

(21) Appl. No.: 18/706,453

(22) PCT Filed: Nov. 4, 2022
(86) PCT No.: PCT/US2022/079266

§ 371 Date: May 1, 2024

Related U.S. Application Data
(60) Provisional application No. 63/275,850, filed on Nov. 4, 2021.

(51) Int. Cl. C07K 14/195; C12N 15/82

(52) U.S. Cl. CPC C07K 14/195; C12N 15/8242

(57) ABSTRACT

The present disclosure provides recombinant polynucleotide comprising a heterologous regulatory element operably linked to a first nucleic acid sequence encoding a cyanobacterial bicarbonate transporter protein. The disclosure also provides expression vectors comprising the recombinant polynucleotides as well as eukaryotic algae and methods of utilizing the eukaryotic algae for improved carbon removal and biomass production.

### CROSS-REFERENCE TO RELATED APPLICATIONS

This application claims the benefit under 35 USC § 119(e) of U.S. Provisional Application Ser. No. 63/275,850, filed on Nov. 4, 2021, the entire disclosure of which is incorporated herein by reference.

### GOVERNMENT INTEREST

This invention was made with government support under grant DE-EE0008514 awarded by the Department of Energy. The government has certain rights in the invention.

### BACKGROUND AND SUMMARY OF THE INVENTION

Algae have an established and valuable place in a number of industries. In view of the ongoing search for renewable alternatives to fossil fuels, global algae product markets are projected to grow at an estimated combined average growth rate of 5.8% per year through 2026, with total revenues projected to reach US $56.5 billion by 2027.

Algaculture produces a broad and diverse range of products, including food additives, nutritional supplements, abrasives, cosmetics, colorants, fertilizers, biofuels, and other naturally occurring algal commercially valuable bioproducts. Algae come in many different forms, including ocean forests of giant kelp to hundreds of thousands of microscopic species. In particular, microalgae are believed to generate almost half of the oxygen in the atmosphere, absorb carbon dioxide (CO₂) at rates 10-50 times greater than terrestrial plants, and provide the base of food for all higher marine.

Further, microalgae can be utilized to produce renewable and sustainable biomass sources for many bioproduct industries, to provide wastewater management, and sequester CO₂ for mitigation of climate change. Microalgae have evolved to optimally grow within certain environmental conditions. Essential variables for efficient microalgae biomass production include water, light, oxygen levels, and carbon dioxide levels.

Microalgae cultivation is performed in both open and closed systems. Open pond cultivation is the oldest and simplest large-scale method industries use to grow microalgae because of its low startup, scaleup, operating, and maintenance costs, as well as the lower energy demand. Alternatively, closed microalgae cultivation systems use less space and produce higher quality products because of better control over cultivation parameters.

Importantly, maintaining sufficient levels of dissolved CO₂ in water environments for optimal microalgae growth can be problematic because excess CO₂ will bubble out of the system and CO₂ reacts with water (H₂O) to form an equilibrium with inorganic bicarbonate (HCO₃⁻). This disadvantage exists for both open and closed cultivation systems, resulting in a reduced amount of dissolved CO₂ available for photosynthesis and thus decreased biomass and bioproduct yields.

To mitigate this problem, industrial growers currently add supplemental CO₂ to open and closed propagation systems. Further, to circumvent this CO₂ limiting problems, current algaculture systems can use CO₂ sparging devices. However, this also results in additional costs due to the requirement of specialized equipment and a ready source of compressed CO₂. Further, the sparging process is inefficient as much of the CO₂ is lost to the atmosphere. Thus, there exists a need for alternative products and systems to provide more efficient means for algaculture.

Accordingly, the present disclosure provides genetically engineered algae that provides improved CO₂ utilization in photosynthesis, thus resulting in increased algal biomass production. The genetically engineered algae of the present disclosure advantageously utilize the CO₂ and HCO₃⁻ endogenously present in the growth environment more efficiently. As a result, the requirement for adding CO₂ is mitigated and biomass production can be increased. The genetically engineered algae can be utilized in both open and closed propagation systems.

Moreover, the genetically engineered algae of the present disclosure also facilitates transport of HCO₃⁻ into the algae, thus redirecting more CO₂ from the growth media into algal cells. In particular, expression of a cyanobacterial bicarbonate transporter protein (BicA) in algae cells allows for the cells to access bicarbonate, resulting in an improved capture of carbon from media and a more efficient process, including higher rates of photosynthesis and growth. This process increases photosynthetic conversion of CO₂ into biomass and bioproducts per unit volume compared to non-modified algae. In addition, inexpensive HCO₃ could be added to growth media and for utilization by the modified algae to provide a further increase in biomass production.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows an exemplary assembled BICA overexpression cassette.

FIG. 2 shows flow cytometry analysis of cells expressing Green Fluorescent Protein (GFP) versus untransformed control cells (WT).

FIG. 3 shows the percent increase in daily biomass accumulation of a BicA-GFP expressing strain (TKK012-216) compared to a control strain without BicA-GFP.

FIG. 4 shows a 29% increase in growth rate of BicA-GFP expressing strain (TKK012-206) relative to a control strain (WT) with two supplemental additions of 0.2 mM HCO₃⁻ per day. Changes in growth rate are based off of twice daily measurements of biomass using OD680 as a proxy; each bar represents one biological replicate.

FIG. 5 shows duplicate measurements of photosynthetic activity of an exemplary BicA-GFP expressing strain (TKK012-20) compared to a control strain (WT) that was not transformed with BicA-GFP fusion protein.

### DETAILED DESCRIPTION

Various embodiments of the invention are described herein as follows. In an illustrative aspect, a recombinant polynucleotide is provided. The recombinant polynucleotide comprises a heterologous regulatory element operably linked to a first nucleic acid sequence encoding a cyanobacterial bicarbonate transporter protein.

In an embodiment, the cyanobacterial bicarbonate transporter protein comprises:

(SEQ. ID NO: 1)
MQITNKIHFRNLQGDLFGGVTAAVIALPMALAFGIASGAGATAGLWGAV
IVGFFAALFGGTPTLISEPTGPMTVVQTAVIASLVAADPDNGLAMAFTV
VMMAGLFQIAFGLLKLGKYVTMMPYTVISGFMSGIGIILVILQLAPFLG
QASPKGGVIGTLQALPNLVSNVRPVETLLALMTVGIIWFMPSRWKKFAP
PQLVALVLGTIISITLFGDLDIRRIGEIQAGLPALQLPVFQADQLQRML
IDAAVLGMLGCIDALLTSVVADSLTRTEHNSNKELVGQGIGNVMSGLFG
GLGGAGATMGTVVNIQSGGRTALSGLIRAMVLLVVILGAAKLAATIPLA
VLAGIAFKVGVDIIDWGFLKRAHHVSIKGALIMYAVIVLTVLVDLIAAV
GIGVFIANILTIDRMSALQSKAVKSISDADDEILLSANEKRWLDEGNGR
VLLFQLSGPMIFGVAKAIAREHNAIQECAAIVFDLSDVPHLGVTASLAL
ENAIEEAAEKGRAVYIVGATGQTKRRLEKLQVFRFVPESNCYDDRSEAL
KDAVLALGPHESEDSPSSSSVQTTY.

SEQ. ID NO: 1 represents the BICA protein sequence from Synechocystis sp. PCC 6803. In another embodiment, the sequence consists essentially of SEQ. ID NO: 1. In yet another embodiment, the sequence consists of SEQ. ID NO: 1.

In an embodiment, the cyanobacterial bicarbonate transporter protein comprises at least 80% sequence identity to the sequence of SEQ ID NO: 1. In an embodiment, the cyanobacterial bicarbonate transporter protein comprises at least 85% sequence identity to the sequence of SEQ ID NO: 1. In an embodiment, the cyanobacterial bicarbonate transporter protein comprises at least 90% sequence identity to the sequence of SEQ ID NO: 1. In an embodiment, the cyanobacterial bicarbonate transporter protein comprises at least 95% sequence identity to the sequence of SEQ ID NO: 1. In an embodiment, the cyanobacterial bicarbonate transporter protein comprises at least 96% sequence identity to the sequence of SEQ ID NO: 1. In an embodiment, the cyanobacterial bicarbonate transporter protein comprises at least 97% sequence identity to the sequence of SEQ ID NO: 1. In an embodiment, the cyanobacterial bicarbonate transporter protein comprises at least 98% sequence identity to the sequence of SEQ ID NO: 1. In an embodiment, the cyanobacterial bicarbonate transporter protein comprises at least 99% sequence identity to the sequence of SEQ ID NO: 1.

In an embodiment, the cyanobacterial bicarbonate transporter protein consists of at least 80% sequence identity to the sequence of SEQ ID NO: 1. In an embodiment, the cyanobacterial bicarbonate transporter protein consists of at least 85% sequence identity to the sequence of SEQ ID NO: 1. In an embodiment, the cyanobacterial bicarbonate transporter protein consists of at least 90% sequence identity to the sequence of SEQ ID NO: 1. In an embodiment, the cyanobacterial bicarbonate transporter protein consists of at least 95% sequence identity to the sequence of SEQ ID NO: 1. In an embodiment, the cyanobacterial bicarbonate transporter protein consists of at least 96% sequence identity to the sequence of SEQ ID NO: 1. In an embodiment, the cyanobacterial bicarbonate transporter protein consists of at least 97% sequence identity to the sequence of SEQ ID NO: 1. In an embodiment, the cyanobacterial bicarbonate transporter protein consists of at least 98% sequence identity to the sequence of SEQ ID NO: 1. In an embodiment, the cyanobacterial bicarbonate transporter protein consists of at least 99% sequence identity to the sequence of SEQ ID NO: 1.

In an embodiment, the cyanobacterial bicarbonate transporter protein is BicA. In an embodiment, the heterologous regulatory element is a promoter that functions in eukaryotic cells. In an embodiment, the promoter is a promoter that functions in eukaryotic algae.

In an embodiment, the cyanobacterial bicarbonate transporter protein is BicA. In an embodiment, the heterologous regulatory element is a promoter that functions in eukaryotic cells. In an embodiment, the promoter is a promoter that functions in eukaryotic algae. In an embodiment, the first nucleic acid sequence is a native cyanobacterial sequence that has been modified to comprise codons optimized for expression in eukaryotic cells. In an embodiment, the first nucleic acid sequence is a cyanobacterial bicarbonate transporter gene from Synechocystis that has been modified to comprise codons optimized for expression in eukaryotic cells.

In an embodiment, the first nucleic acid sequence comprises:

(SEQ. ID NO: 2)
ATGCAAATAACTAACAAAATTCATTTTAGGAACCTGCAGGGGGACCTTTTTGGCGG
GGTTACAGCGGCGGTTATTGCCCTGCCCATGGCCTTAGCCTTCGGGATTGCTTCCGG
AGCAGGGGCTACGGCCGGACTCTGGGGGGCGGTGATCGTAGGGTTTTTCGCGGCCT
TATTTGGCGGCACCCCCACCTTAATTTCCGAACCGACTGGGCCCATGACGGTGGTG
CAAACGGCGGTTATTGCTAGTTTAGTGGCGGCAGATCCCGACAATGGCTTGGCCAT
GGCCTTCACTGTGGTAATGATGGCGGGGTTGTTCCAGATTGCCTTTGGTCTGCTCAA
ATTGGGCAAATATGTCACCATGATGCCCTACACAGTCATTTCCGGCTTTATGTCCGG
CATTGGGATTATTTTGGTGATTTTGCAACTGGCTCCCTTTCTTGGCCAAGCTAGTCC
CAAGGGAGGGGTAATCGGCACCCTCCAGGCCCTCCCTAACCTAGTAAGCAATGTCA
GGCCGGTGGAAACCCTATTGGCGCTCATGACGGTGGGCATTATTTGGTTTATGCCTT
CCCGTTGGAAAAAGTTTGCTCCGCCCCAATTGGTGGCTTTAGTGTTGGGGACAATT
ATTTCCATCACCCTATTTGGCGATCTGGATATCCGTCGCATTGGGGAAATTCAGGCC
GGTTTGCCCGCTCTACAGCTACCAGTGTTTCAGGCTGATCAATTACAGAGAATGCT
GATTGATGCGGCTGTTCTGGGAATGCTGGGCTGTATTGATGCCCTCCTGACTTCGGT
GGTGGCTGATAGCTTGACCCGCACAGAACATAACTCCAACAAGGAATTAGTCGGCC
AGGGCATCGGCAATGTAATGTCCGGTTTATTTGGTGGCTTGGGGGGAGCTGGGGCC
ACCATGGGGACGGTGGTAAATATCCAGTCCGGGGGACGCACAGCTCTGTCTGGCTT
GATCCGGGCGATGGTGTTGCTGGTGGTAATTTTAGGCGCAGCTAAATTGGCGGCTA
CCATTCCCCTAGCCGTATTGGCTGGTATTGCGTTCAAAGTTGGGGTGGACATTATTG
ATTGGGGGTTCCTCAAGCGGGCTCACCATGTCTCCATCAAAGGGGCCTTGATTATG
TATGCCGTCATTGTCCTGACGGTGTTGGTGGATTTAATTGCGGCAGTAGGTATTGGT
GTATTTATTGCCAATATTCTCACCATTGACCGTATGAGTGCGTTGCAGTCCAAAGCT
GTGAAAAGTATTAGCGATGCCGACGACGAAATTCTCCTTTCCGCCAATGAGAAACG
TTGGCTAGATGAGGGCAATGGCCGGGTCTTGCTTTTCCAACTCAGTGGCCCAATGA
TTTTTGGGGTGGCCAAGGCGATCGCCAGGGAACATAATGCCATTCAAGAATGTGCC
GCCATTGTTTTTGATCTGAGCGATGTGCCCCATTTGGGAGTAACCGCTTCCCTGGCC
CTGGAAAATGCCATTGAAGAAGCGGCGGAAAAAGGTCGGGCCGTTTACATTGTGG
GGGCAACAGGGCAAACCAAGCGACGCTTGGAAAAATTGCAAGTGTTCCGCTTTGTT
CCTGAAAGTAATTGCTATGACGACCGTTCTGAAGCTCTCAAGGACGCTGTCCTAGC
TTTGGGACCTCATGAAAGTGAGGACTCCCCTTCCAGTTCTTCCGTCCAGACCACATA
CTGA.

SEQ. ID NO: 2 represents the BICA coding sequence from Synechocystis sp. PCC 6803. In another embodiment, the first nucleic acid sequence consists essentially of SEQ. ID NO: 2. In yet another embodiment, the first nucleic acid sequence consists of SEQ. ID NO: 2.

In an embodiment, the first nucleic acid sequence comprises a sequence having at least 80% sequence identity to SEQ ID NO: 2. In an embodiment, the first nucleic acid sequence comprises a sequence having at least 85% sequence identity to SEQ ID NO: 2. In an embodiment, the first nucleic acid sequence comprises a sequence having at least 90% sequence identity to SEQ ID NO: 2. In an embodiment, the first nucleic acid sequence comprises a sequence having at least 95% sequence identity to SEQ ID NO: 2. In an embodiment, the first nucleic acid sequence comprises a sequence having at least 96% sequence identity to SEQ ID NO: 2. In an embodiment, the first nucleic acid sequence comprises a sequence having at least 97% sequence identity to SEQ ID NO: 2. In an embodiment, the first nucleic acid sequence comprises a sequence having at least 98% sequence identity to SEQ ID NO: 2. In an embodiment, the first nucleic acid sequence comprises a sequence having at least 99% sequence identity to SEQ ID NO: 2.

In an embodiment, the first nucleic acid sequence consists of a sequence having at least 80% sequence identity to SEQ ID NO: 2. In an embodiment, the first nucleic acid sequence consists of a sequence having at least 85% sequence identity to SEQ ID NO: 2. In an embodiment, the first nucleic acid sequence consists of a sequence having at least 90% sequence identity to SEQ ID NO: 2. In an embodiment, the first nucleic acid sequence consists of a sequence having at least 95% sequence identity to SEQ ID NO: 2. In an embodiment, the first nucleic acid sequence consists of a sequence having at least 96% sequence identity to SEQ ID NO: 2. In an embodiment, the first nucleic acid sequence consists of a sequence having at least 97% sequence identity to SEQ ID NO: 2. In an embodiment, the first nucleic acid sequence consists of a sequence having at least 98% sequence identity to SEQ ID NO: 2. In an embodiment, the first nucleic acid sequence consists of a sequence having at least 99% sequence identity to SEQ ID NO: 2.

In an embodiment, the first nucleic acid sequence comprises:

(SEQ. ID NO: 3)
ATGCAGATCACGAACAAGATTCACTTCCGAAACTTGCAGGGTGACTTGTTTGGTGG
TGTCACGGCTGCCGTCATTGCCTTGCCTATGGCCTTGGCCTTCGGTATTGCGTCCGG
CGCTGGTGCCACGGCTGGTTTGTGGGGGGCTGTGATCGTCGGTTTCTTCGCGGCCTT
GTTTGGCGGCACCCCTACCTTGATTTCCGAGCCTACCGGCCCCATGACGGTCGTGC
AGACGGCGGTGATTGCGTCCTTGGTGGCGGCCGACCCCGACAACGGCTTGGCCATG
GCCTTTACGGTGGTCATGATGGCGGGGTTGTTCCAGATTGCCTTCGGCTTGCTCAAG
TTGGGCAAGTACGTCACCATGATGCCCTACACGGTCATTTCCGGCTTCATGTCCGGC
ATCGGGATCATTTTGGTGATCTTGCAGCTCGCCCCCTTCCTCGGCCAGGCCAGCCCC
AAGGGCGGGGTCATCGGCACCTTGCAGGCCCTCCCTAACCTCGTCAGCAACGTCCG
GCCCGTCGAGACGCTCTTGGCGCTCATGACGGTGGGCATTATCTGGTTCATGCCTTC
CCGCTGGAAGAAGTTCGCGCCTCCCCAGTTGGTGGCCTTGGTGTTGGGGACCATCA
TTTCCATCACCCTCTTCGGCGACCTGGACATCCGCCGCATTGGGGAGATTCAGGCT
GGTTTGCCTGCCCTCCAGTTGCCTGTGTTTCAGGCGGACCAGTTGCAGCGCATGTTG
ATTGACGCGGCTGTGTTGGGCATGTTGGGCTGCATTGACGCTCTCTTGACTTCCGTG
GTGGCTGACAGCTTGACCCGCACGGAGCACAACTCCAACAAGGAGTTGGTCGGCC
AGGGCATCGGCAACGTGATGTCCGGCTTGTTCGGCGGCTTGGGGGGCGCTGGTGCA
ACCATGGGGACGGTGGTCAACATTCAGTCCGGTGGCCGAACGGCATTGTCCGGCTT
GATCCGGGCGATGGTGTTGCTGGTGGTCATTTTGGGCGCCGCGAAGTTGGCGGCCA
CCATCCCCCTCGCCGTGTTGGCCGGCATCGCGTTCAAGGTCGGGGTGGACATCATT
GACTGGGGGTTCCTCAAGCGGGCGCACCACGTCTCCATCAAGGGCGCCTTGATTAT
GTACGCTGTCATTGTCTTGACGGTGTTGGTGGACTTGATTGCGGCTGTCGGCATTGG
TGTGTTTATTGCCAACATCTTGACCATCGACCGAATGAGCGCGTTGCAGTCCAAGG
CCGTGAAGTCCATCAGCGATGCCGATGACGAGATTCTCTTGTCCGCTAACGAGAAG
CGCTGGTTGGACGAGGGCAACGGCCGGGTCTTGCTCTTTCAGTTGAGCGGCCCCAT
GATTTTTGGTGTGGCCAAGGCGATTGCCCGCGAGCACAACGCCATTCAGGAGTGCG
CCGCCATCGTGTTCGACTTGAGCGACGTGCCCCACTTGGGCGTGACTGCTTCCTTGG
CCTTGGAGAACGCCATTGAGGAGGCCGCCGAGAAGGGTCGGGCTGTTTACATCGTG
GGGGCCACCGGGCAGACCAAGCGCCGCTTGGAGAAGTTGCAGGTGTTTCGCTTCGT
GCCTGAGTCCAACTGCTACGACGACCGCTCCGAGGCCCTCAAGGACGCGGTGCTCG
CTTTGGGTCCTCACGAGTCCGAGGATTCCCCTTCCAGCTCCAGCGTCCAGACCACGT
ACTAA

SEQ. ID NO: 3 represents the BICA coding sequence codon optimized for N. oceanica. In another embodiment, the first nucleic acid sequence consists essentially of SEQ. ID NO: 3. In yet another embodiment, the first nucleic acid sequence consists of SEQ. ID NO: 3.

In an embodiment, the first nucleic acid sequence comprises a sequence having at least 80% sequence identity to SEQ ID NO: 3. In an embodiment, the first nucleic acid sequence comprises a sequence having at least 85% sequence identity to SEQ ID NO: 3. In an embodiment, the first nucleic acid sequence comprises a sequence having at least 90% sequence identity to SEQ ID NO: 3. In an embodiment, the first nucleic acid sequence comprises a sequence having at least 95% sequence identity to SEQ ID NO: 3. In an embodiment, the first nucleic acid sequence comprises a sequence having at least 96% sequence identity to SEQ ID NO: 3. In an embodiment, the first nucleic acid sequence comprises a sequence having at least 97% sequence identity to SEQ ID NO: 3. In an embodiment, the first nucleic acid sequence comprises a sequence having at least 98% sequence identity to SEQ ID NO: 3. In an embodiment, the first nucleic acid sequence comprises a sequence having at least 99% sequence identity to SEQ ID NO: 3.

In an embodiment, the first nucleic acid sequence consists of a sequence having at least 80% sequence identity to SEQ ID NO: 3. In an embodiment, the first nucleic acid sequence consists of a sequence having at least 85% sequence identity to SEQ ID NO: 3. In an embodiment, the first nucleic acid sequence consists of a sequence having at least 90% sequence identity to SEQ ID NO: 3. In an embodiment, the first nucleic acid sequence consists of a sequence having at least 95% sequence identity to SEQ ID NO: 3. In an embodiment, the first nucleic acid sequence consists of a sequence having at least 96% sequence identity to SEQ ID NO: 3. In an embodiment, the first nucleic acid sequence consists of a sequence having at least 97% sequence identity to SEQ ID NO: 3. In an embodiment, the first nucleic acid sequence consists of a sequence having at least 98% sequence identity to SEQ ID NO: 3. In an embodiment, the first nucleic acid sequence consists of a sequence having at least 99% sequence identity to SEQ ID NO: 3.

In an embodiment, the recombinant polynucleotide further comprises a second nucleic acid sequence encoding a marker. In an embodiment, the second nucleic acid sequence is transcriptionally linked to the first nucleic acid sequence and the polynucleotide encodes a fusion peptide comprising the cyanobacterial bicarbonate transporter protein and the marker.

In an embodiment, the marker is a fluorescent marker. In an embodiment, the fluorescent marker is Green Fluorescent Protein (GFP). In an embodiment, the encoded polypeptide comprises GFP fused to the N-terminus of the cyanobacterial bicarbonate transporter protein. In an embodiment, the fluorescent marker is Yellow Fluorescent Protein (YFP). In an embodiment, the encoded polypeptide comprises YFP fused to the N-terminus of the cyanobacterial bicarbonate transporter protein. In an embodiment, the fluorescent marker is a phycobiliprotein derivative.

In an illustrative aspect, an expression vector is provided. The expression vector comprises a recombinant polynucleotide of any embodiment described herein and a selectable marker gene.

In an embodiment, the selectable marker gene encodes for antibiotic resistance. In an embodiment, the selectable marker gene is neomycin (Neo). In an embodiment, the selectable marker gene is bleomycin (Ble). In an embodiment, the selectable marker gene is blasticidin (Bsr/Bsd).

In an embodiment, the selectable marker gene confers neomycin resistance. In an embodiment, the selectable marker gene confers bleomycin resistance. In an embodiment, the selectable marker gene confers blasticidin resistance.

In an illustrative aspect, a eukaryotic alga is provided. The eukaryotic alga comprises an expression vector of any embodiment described herein.

In an embodiment, the eukaryotic alga is a Nannochloropsis species. In an embodiment, the eukaryotic alga is Nannochloropsis oceanica. In an embodiment, the eukaryotic alga is Nannochloropsis gaditana. In an embodiment, the eukaryotic alga is Nannochloropsis oculata.

In an embodiment, the eukaryotic alga is Picochlorum species. In an embodiment, the eukaryotic alga is Chlorella species. In an embodiment, the eukaryotic alga is Desmodesmus species. In an embodiment, the eukaryotic alga is Monorhaphidium species. In an embodiment, the eukaryotic alga is Phaeodactylum species. In an embodiment, the eukaryotic alga is Cyclotella species. In an embodiment, the eukaryotic alga is Scenedesmus species. In an embodiment, the eukaryotic alga is Thalassiosira species. In an embodiment, the eukaryotic alga is Nannochloris species.

In an illustrative aspect, a method of removing carbon from a substance is provided. The method comprises the step of contacting the eukaryotic algae of any embodiment described herein to the substance, wherein the substance comprises carbon, and wherein the eukaryotic algae removes carbon from the substance.

In an embodiment, the substance is liquid. In an embodiment, the liquid is growth media.

In an embodiment, the carbon removed from the substance is bicarbonate. In an embodiment, the bicarbonate is a product of carbon dioxide.

In an embodiment, the carbon removed from the substance is carbon dioxide. In an embodiment, the removal of carbon corresponds to increased carbon dioxide utilization of the eukaryotic algae. In an embodiment, the removal of carbon corresponds to improved photosynthesis of the eukaryotic algae. In an embodiment, the removal of carbon corresponds to increased biomass production by the eukaryotic algae. In an embodiment, the removal of carbon by the eukaryotic algae is higher than an algae not modified by a recombinant polynucleotide described herein.

In an illustrative aspect, a method of increasing biomass production is provided. The method comprises the step of culturing the eukaryotic algae of any embodiment described herein, wherein the eukaryotic algae produces more biomass compared to an algae not modified by a recombinant polynucleotide described herein.

In an embodiment, the increase in biomass production corresponds to increased carbon dioxide utilization of the eukaryotic algae. In an embodiment, the increase in biomass production corresponds to improved photosynthesis of the eukaryotic algae. In an embodiment, the increase in biomass production corresponds to increased carbon removal by the eukaryotic algae.

The following numbered embodiments are contemplated and are non-limiting:
1. A recombinant polynucleotide comprising a heterologous regulatory element operably linked to a first nucleic acid sequence encoding a cyanobacterial bicarbonate transporter protein.
2. The recombinant polynucleotide of clause 1, any other suitable clause, or any combination of suitable clauses, wherein the cyanobacterial bicarbonate transporter protein comprises SEQ ID NO: 1.
3. The recombinant polynucleotide of clause 1, any other suitable clause, or any combination of suitable clauses, wherein the cyanobacterial bicarbonate transporter protein comprises at least 80% sequence identity to the sequence of SEQ ID NO: 1.
4. The recombinant polynucleotide of clause 1, any other suitable clause, or any combination of suitable clauses, wherein the cyanobacterial bicarbonate transporter protein comprises at least 85% sequence identity to the sequence of SEQ ID NO: 1.
5. The recombinant polynucleotide of clause 1, any other suitable clause, or any combination of suitable clauses, wherein the cyanobacterial bicarbonate transporter protein comprises at least 90% sequence identity to the sequence of SEQ ID NO: 1.
6. The recombinant polynucleotide of clause 1, any other suitable clause, or any combination of suitable clauses, wherein the cyanobacterial bicarbonate transporter protein comprises at least 95% sequence identity to the sequence of SEQ ID NO: 1.
7. The recombinant polynucleotide of clause 1, any other suitable clause, or any combination of suitable clauses, wherein the cyanobacterial bicarbonate transporter protein comprises at least 96% sequence identity to the sequence of SEQ ID NO: 1.
8. The recombinant polynucleotide of clause 1, any other suitable clause, or any combination of suitable clauses, wherein the cyanobacterial bicarbonate transporter protein comprises at least 97% sequence identity to the sequence of SEQ ID NO: 1.
9. The recombinant polynucleotide of clause 1, any other suitable clause, or any combination of suitable clauses, wherein the cyanobacterial bicarbonate transporter protein comprises at least 98% sequence identity to the sequence of SEQ ID NO: 1.
10. The recombinant polynucleotide of clause 1, any other suitable clause, or any combination of suitable clauses, wherein the cyanobacterial bicarbonate transporter protein comprises at least 99% sequence identity to the sequence of SEQ ID NO: 1.
11. The recombinant polynucleotide of clause 1, any other suitable clause, or any combination of suitable clauses, wherein the cyanobacterial bicarbonate transporter protein consists of at least 80% sequence identity to the sequence of SEQ ID NO: 1.
12. The recombinant polynucleotide of clause 1, any other suitable clause, or any combination of suitable clauses, wherein the cyanobacterial bicarbonate transporter protein consists of at least 85% sequence identity to the sequence of SEQ ID NO: 1.
13. The recombinant polynucleotide of clause 1, any other suitable clause, or any combination of suitable clauses, wherein the cyanobacterial bicarbonate transporter protein consists of at least 90% sequence identity to the sequence of SEQ ID NO: 1.
14. The recombinant polynucleotide of clause 1, any other suitable clause, or any combination of suitable clauses, wherein the cyanobacterial bicarbonate transporter protein consists of at least 95% sequence identity to the sequence of SEQ ID NO: 1.
15. The recombinant polynucleotide of clause 1, any other suitable clause, or any combination of suitable clauses, wherein the cyanobacterial bicarbonate transporter protein consists of at least 96% sequence identity to the sequence of SEQ ID NO: 1.
16. The recombinant polynucleotide of clause 1, any other suitable clause, or any combination of suitable clauses, wherein the cyanobacterial bicarbonate transporter protein consists of at least 97% sequence identity to the sequence of SEQ ID NO: 1.
17. The recombinant polynucleotide of clause 1, any other suitable clause, or any combination of suitable clauses, wherein the cyanobacterial bicarbonate transporter protein consists of at least 98% sequence identity to the sequence of SEQ ID NO: 1.
18. The recombinant polynucleotide of clause 1, any other suitable clause, or any combination of suitable clauses, wherein the cyanobacterial bicarbonate transporter protein consists of at least 99% sequence identity to the sequence of SEQ ID NO: 1.
19. The recombinant polynucleotide of clause 1, any other suitable clause, or any combination of suitable clauses, wherein the cyanobacterial bicarbonate transporter protein is BicA.
20. The recombinant polynucleotide of clause 1, any other suitable clause, or any combination of suitable clauses, wherein the heterologous regulatory element is a promoter that functions in eukaryotic cells.
21. The recombinant polynucleotide of clause 1, any other suitable clause, or any combination of suitable clauses, wherein the promoter is a promoter that functions in eukaryotic algae.
22. The recombinant polynucleotide of clause 1, any other suitable clause, or any combination of suitable clauses, wherein the first nucleic acid sequence is a native cyanobacterial sequence that has been modified to comprise codons optimized for expression in eukaryotic cells.
23. The recombinant polynucleotide of clause 22, any other suitable clause, or any combination of suitable clauses, wherein the first nucleic acid sequence is a cyanobacterial bicarbonate transporter gene from Synechocystis that has been modified to comprise codons optimized for expression in eukaryotic cells.
24. The recombinant polynucleotide of clause 1, any other suitable clause, or any combination of suitable clauses, wherein the first nucleic acid sequence comprises SEQ ID NO: 2.
25. The recombinant polynucleotide of clause 1, any other suitable clause, or any combination of suitable clauses, wherein the first nucleic acid sequence comprises a sequence having at least 80% sequence identity to SEQ ID NO: 2.
26. The recombinant polynucleotide of clause 1, any other suitable clause, or any combination of suitable clauses, wherein the first nucleic acid sequence comprises a sequence having at least 85% sequence identity to SEQ ID NO: 2.
27. The recombinant polynucleotide of clause 1, any other suitable clause, or any combination of suitable clauses, wherein the first nucleic acid sequence comprises a sequence having at least 90% sequence identity to SEQ ID NO: 2.
28. The recombinant polynucleotide of clause 1, any other suitable clause, or any combination of suitable clauses, wherein the first nucleic acid sequence comprises a sequence having at least 95% sequence identity to SEQ ID NO: 2.
29. The recombinant polynucleotide of clause 1, any other suitable clause, or any combination of suitable clauses, wherein the first nucleic acid sequence comprises a sequence having at least 96% sequence identity to SEQ ID NO: 2.
30. The recombinant polynucleotide of clause 1, any other suitable clause, or any combination of suitable clauses, wherein the first nucleic acid sequence comprises a sequence having at least 97% sequence identity to SEQ ID NO: 2.
31. The recombinant polynucleotide of clause 1, any other suitable clause, or any combination of suitable clauses, wherein the first nucleic acid sequence comprises a sequence having at least 98% sequence identity to SEQ ID NO: 2.
32. The recombinant polynucleotide of clause 1, any other suitable clause, or any combination of suitable clauses, wherein the first nucleic acid sequence comprises a sequence having at least 99% sequence identity to SEQ ID NO: 2.
33. The recombinant polynucleotide of clause 1, any other suitable clause, or any combination of suitable clauses, wherein the first nucleic acid sequence consists of a sequence having at least 80% sequence identity to SEQ ID NO: 2.
34. The recombinant polynucleotide of clause 1, any other suitable clause, or any combination of suitable clauses, wherein the first nucleic acid sequence consists of a sequence having at least 85% sequence identity to SEQ ID NO: 2.
35. The recombinant polynucleotide of clause 1, any other suitable clause, or any combination of suitable clauses, wherein the first nucleic acid sequence consists of a sequence having at least 90% sequence identity to SEQ ID NO: 2.
36. The recombinant polynucleotide of clause 1, any other suitable clause, or any combination of suitable clauses, wherein the first nucleic acid sequence consists of a sequence having at least 95% sequence identity to SEQ ID NO: 2.
37. The recombinant polynucleotide of clause 1, any other suitable clause, or any combination of suitable clauses, wherein the first nucleic acid sequence consists of a sequence having at least 96% sequence identity to SEQ ID NO: 2.
38. The recombinant polynucleotide of clause 1, any other suitable clause, or any combination of suitable clauses, wherein the first nucleic acid sequence consists of a sequence having at least 97% sequence identity to SEQ ID NO: 2.
39. The recombinant polynucleotide of clause 1, any other suitable clause, or any combination of suitable clauses, wherein the first nucleic acid sequence consists of a sequence having at least 98% sequence identity to SEQ ID NO: 2.
40. The recombinant polynucleotide of clause 1, any other suitable clause, or any combination of suitable clauses, wherein the first nucleic acid sequence consists of a sequence having at least 99% sequence identity to SEQ ID NO: 2.
41. The recombinant polynucleotide of clause 1, any other suitable clause, or any combination of suitable clauses, wherein the first nucleic acid sequence comprises SEQ ID NO: 3.
42. The recombinant polynucleotide of clause 1, any other suitable clause, or any combination of suitable clauses, wherein the first nucleic acid sequence comprises a sequence having at least 80% sequence identity to SEQ ID NO: 3.
43. The recombinant polynucleotide of clause 1, any other suitable clause, or any combination of suitable clauses, wherein the first nucleic acid sequence comprises a sequence having at least 85% sequence identity to SEQ ID NO: 3.
44. The recombinant polynucleotide of clause 1, any other suitable clause, or any combination of suitable clauses, wherein the first nucleic acid sequence comprises a sequence having at least 90% sequence identity to SEQ ID NO: 3.
45. The recombinant polynucleotide of clause 1, any other suitable clause, or any combination of suitable clauses, wherein the first nucleic acid sequence comprises a sequence having at least 95% sequence identity to SEQ ID NO: 3.
46. The recombinant polynucleotide of clause 1, any other suitable clause, or any combination of suitable clauses, wherein the first nucleic acid sequence comprises a sequence having at least 96% sequence identity to SEQ ID NO: 3.
47. The recombinant polynucleotide of clause 1, any other suitable clause, or any combination of suitable clauses, wherein the first nucleic acid sequence comprises a sequence having at least 97% sequence identity to SEQ ID NO: 3.
48. The recombinant polynucleotide of clause 1, any other suitable clause, or any combination of suitable clauses, wherein the first nucleic acid sequence comprises a sequence having at least 98% sequence identity to SEQ ID NO: 3.
49. The recombinant polynucleotide of clause 1, any other suitable clause, or any combination of suitable clauses, wherein the first nucleic acid sequence comprises a sequence having at least 99% sequence identity to SEQ ID NO: 3.
50. The recombinant polynucleotide of clause 1, any other suitable clause, or any combination of suitable clauses, wherein the first nucleic acid sequence consists of a sequence having at least 80% sequence identity to SEQ ID NO: 3.
51. The recombinant polynucleotide of clause 1, any other suitable clause, or any combination of suitable clauses, wherein the first nucleic acid sequence consists of a sequence having at least 85% sequence identity to SEQ ID NO: 3.
52. The recombinant polynucleotide of clause 1, any other suitable clause, or any combination of suitable clauses, wherein the first nucleic acid sequence consists of a sequence having at least 90% sequence identity to SEQ ID NO: 3.
53. The recombinant polynucleotide of clause 1, any other suitable clause, or any combination of suitable clauses, wherein the first nucleic acid sequence consists of a sequence having at least 95% sequence identity to SEQ ID NO: 3.
54. The recombinant polynucleotide of clause 1, any other suitable clause, or any combination of suitable clauses, wherein the first nucleic acid sequence consists of a sequence having at least 96% sequence identity to SEQ ID NO: 3.
55. The recombinant polynucleotide of clause 1, any other suitable clause, or any combination of suitable clauses, wherein the first nucleic acid sequence consists of a sequence having at least 97% sequence identity to SEQ ID NO: 3.
56. The recombinant polynucleotide of clause 1, any other suitable clause, or any combination of suitable clauses, wherein the first nucleic acid sequence consists of a sequence having at least 98% sequence identity to SEQ ID NO: 3.
57. The recombinant polynucleotide of clause 1, any other suitable clause, or any combination of suitable clauses, wherein the first nucleic acid sequence consists of a sequence having at least 99% sequence identity to SEQ ID NO: 3.
58. The recombinant polynucleotide of clause 1, any other suitable clause, or any combination of suitable clauses, further comprising a second nucleic acid sequence encoding a marker.
59. The recombinant polynucleotide of clause 58, any other suitable clause, or any combination of suitable clauses, wherein the second nucleic acid sequence is transcriptionally linked to the first nucleic acid sequence and the polynucleotide encodes a fusion peptide comprising the cyanobacterial bicarbonate transporter protein and the marker.
60. The recombinant polynucleotide of clause 58, any other suitable clause, or any combination of suitable clauses, wherein the marker is a fluorescent marker.
61. The recombinant polynucleotide of clause 60, any other suitable clause, or any combination of suitable clauses, wherein the fluorescent marker is Green Fluorescent Protein (GFP).
62. The recombinant polynucleotide of clause 61, any other suitable clause, or any combination of suitable clauses, wherein the encoded polypeptide comprises GFP fused to the N-terminus of the cyanobacterial bicarbonate transporter protein.
63. The recombinant polynucleotide of clause 60, any other suitable clause, or any combination of suitable clauses, wherein the fluorescent marker is Yellow Fluorescent Protein (YFP).
64. The recombinant polynucleotide of clause 63, any other suitable clause, or any combination of suitable clauses, wherein the encoded polypeptide comprises YFP fused to the N-terminus of the cyanobacterial bicarbonate transporter protein.
65. The recombinant polynucleotide of clause 60, any other suitable clause, or any combination of suitable clauses, wherein the fluorescent marker is a phycobiliprotein derivative.
66. An expression vector comprising a recombinant polynucleotide of any one of clauses 1 to 65 and a selectable marker gene.
67. The expression vector of clause 66, any other suitable clause, or any combination of suitable clauses, wherein the selectable marker gene encodes for antibiotic resistance.
68. The expression vector of clause 67, any other suitable clause, or any combination of suitable clauses, wherein the selectable marker gene is neomycin (Neo).
69. The expression vector of clause 67, any other suitable clause, or any combination of suitable clauses, wherein the selectable marker gene is bleomycin (Ble).
70. The expression vector of clause 67, any other suitable clause, or any combination of suitable clauses, wherein the selectable marker gene is blasticidin (Bsr/Bsd).
71. The expression vector of clause 67, any other suitable clause, or any combination of suitable clauses, wherein the selectable marker gene confers neomycin resistance.
72. The expression vector of clause 67, any other suitable clause, or any combination of suitable clauses, wherein the selectable marker gene confers bleomycin resistance.
73. The expression vector of clause 67, any other suitable clause, or any combination of suitable clauses, wherein the selectable marker gene confers blasticidin resistance.
74. A eukaryotic alga comprising the expression vector of any one of clauses 1 to 73.
75. The eukaryotic alga of clause 74, any other suitable clause, or any combination of suitable clauses, wherein the eukaryotic alga is a Nannochloropsis species.
76. The eukaryotic alga of clause 74, any other suitable clause, or any combination of suitable clauses, wherein the eukaryotic alga is Nannochloropsis oceanica.
77. The eukaryotic alga of clause 74, any other suitable clause, or any combination of suitable clauses, wherein the eukaryotic alga is Nannochloropsis gaditana.
78. The eukaryotic alga of clause 74, any other suitable clause, or any combination of suitable clauses, wherein the eukaryotic alga is Nannochloropsis oculata.
79. The eukaryotic alga of clause 74, any other suitable clause, or any combination of suitable clauses, wherein the eukaryotic alga is Picochlorum species.
80. The eukaryotic alga of clause 74, any other suitable clause, or any combination of suitable clauses, wherein the eukaryotic alga is Chlorella species.
81. The eukaryotic alga of clause 74, any other suitable clause, or any combination of suitable clauses, wherein the eukaryotic alga is Desmodesmus species.
82. The eukaryotic alga of clause 74, any other suitable clause, or any combination of suitable clauses, wherein the eukaryotic alga is Monorhaphidium species.
83. The eukaryotic alga of clause 74, any other suitable clause, or any combination of suitable clauses, wherein the eukaryotic alga is Phaeodactylum species.
84. The eukaryotic alga of clause 74, any other suitable clause, or any combination of suitable clauses, wherein the eukaryotic alga is Cyclotella species.
85. The eukaryotic alga of clause 74, any other suitable clause, or any combination of suitable clauses, wherein the eukaryotic alga is Scenedesmus species.
86. The eukaryotic alga of clause 74, any other suitable clause, or any combination of suitable clauses, wherein the eukaryotic alga is Thalassiosira species.
87. The eukaryotic alga of clause 74, any other suitable clause, or any combination of suitable clauses, wherein the eukaryotic alga is Nannochloris species.
88. A method of removing carbon from a substance, said method comprising the step of contacting the eukaryotic algae of any one of clauses 74 to 87 to the substance, wherein the substance comprises carbon, and wherein the eukaryotic algae removes carbon from the substance.
89. The method of clause 88, any other suitable clause, or any combination of suitable clauses, wherein the substance is liquid.
90. The method of clause 88, any other suitable clause, or any combination of suitable clauses, wherein the liquid is growth media.
91. The method of clause 88, any other suitable clause, or any combination of suitable clauses, wherein the carbon removed from the substance is bicarbonate.
92. The method of clause 91, any other suitable clause, or any combination of suitable clauses, wherein the bicarbonate is a product of carbon dioxide.
93. The method of clause 88, any other suitable clause, or any combination of suitable clauses, wherein the carbon removed from the substance is carbon dioxide.
94. The method of clause 88, any other suitable clause, or any combination of suitable clauses, wherein the removal of carbon corresponds to increased carbon dioxide utilization of the eukaryotic algae.
95. The method of clause 88, any other suitable clause, or any combination of suitable clauses, wherein the removal of carbon corresponds to improved photosynthesis of the eukaryotic algae.
96. The method of clause 88, any other suitable clause, or any combination of suitable clauses, wherein the removal of carbon corresponds to increased biomass production by the eukaryotic algae.
97. The method of clause 88, any other suitable clause, or any combination of suitable clauses, wherein the removal of carbon by the eukaryotic algae is higher than an algae not modified by the recombinant polynucleotide of any one of the above clauses.
98. A method of increasing biomass production, said method comprising the step of culturing the eukaryotic algae of any one of clauses 74 to 87, wherein the eukaryotic algae produces more biomass compared to an algae not modified by the recombinant polynucleotide of any one of the above clauses.
99. The method of clause 98, any other suitable clause, or any combination of suitable clauses, wherein the increase in biomass production corresponds to increased carbon dioxide utilization of the eukaryotic algae.
100. The method of clause 98, any other suitable clause, or any combination of suitable clauses, wherein the increase in biomass production corresponds to improved photosynthesis of the eukaryotic algae.
101. The method of clause 98, any other suitable clause, or any combination of suitable clauses, wherein the increase in biomass production corresponds to increased carbon removal by the eukaryotic algae.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

### Example 1

### BicA Sequences

In the instant example, a BicA sequence was obtained from Synechocystis sp. PCC 6803. The sequence can be codon optimized for utilization with the present disclosure.

For instance, the BicA sequence can be codon optimized for N. oceanica. In the instant example, the sequence was codon optimized for use with N. oceanica strain CCAP849/10. The codon optimized sequence was determined to be as follows:

(SEQ. ID NO: 3)
ATGCAGATCACGAACAAGATTCACTTCCGAAACTTGCAGGGTGACTTGTTTGGTGG
TGTCACGGCTGCCGTCATTGCCTTGCCTATGGCCTTGGCCTTCGGTATTGCGTCCGG
CGCTGGTGCCACGGCTGGTTTGTGGGGGGCTGTGATCGTCGGTTTCTTCGCGGCCTT
GTTTGGCGGCACCCCTACCTTGATTTCCGAGCCTACCGGCCCCATGACGGTCGTGC
AGACGGCGGTGATTGCGTCCTTGGTGGCGGCCGACCCCGACAACGGCTTGGCCATG
GCCTTTACGGTGGTCATGATGGCGGGGTTGTTCCAGATTGCCTTCGGCTTGCTCAAG
TTGGGCAAGTACGTCACCATGATGCCCTACACGGTCATTTCCGGCTTCATGTCCGGC
ATCGGGATCATTTTGGTGATCTTGCAGCTCGCCCCCTTCCTCGGCCAGGCCAGCCCC
AAGGGCGGGGTCATCGGCACCTTGCAGGCCCTCCCTAACCTCGTCAGCAACGTCCG
GCCCGTCGAGACGCTCTTGGCGCTCATGACGGTGGGCATTATCTGGTTCATGCCTTC
CCGCTGGAAGAAGTTCGCGCCTCCCCAGTTGGTGGCCTTGGTGTTGGGGACCATCA
TTTCCATCACCCTCTTCGGCGACCTGGACATCCGCCGCATTGGGGAGATTCAGGCT
GGTTTGCCTGCCCTCCAGTTGCCTGTGTTTCAGGCGGACCAGTTGCAGCGCATGTTG
ATTGACGCGGCTGTGTTGGGCATGTTGGGCTGCATTGACGCTCTCTTGACTTCCGTG
GTGGCTGACAGCTTGACCCGCACGGAGCACAACTCCAACAAGGAGTTGGTCGGCC
AGGGCATCGGCAACGTGATGTCCGGCTTGTTCGGCGGCTTGGGGGGCGCTGGTGCA
ACCATGGGGACGGTGGTCAACATTCAGTCCGGTGGCCGAACGGCATTGTCCGGCTT
GATCCGGGCGATGGTGTTGCTGGTGGTCATTTTGGGCGCCGCGAAGTTGGCGGCCA
CCATCCCCCTCGCCGTGTTGGCCGGCATCGCGTTCAAGGTCGGGGTGGACATCATT
GACTGGGGGTTCCTCAAGCGGGCGCACCACGTCTCCATCAAGGGCGCCTTGATTAT
GTACGCTGTCATTGTCTTGACGGTGTTGGTGGACTTGATTGCGGCTGTCGGCATTGG
TGTGTTTATTGCCAACATCTTGACCATCGACCGAATGAGCGCGTTGCAGTCCAAGG
CCGTGAAGTCCATCAGCGATGCCGATGACGAGATTCTCTTGTCCGCTAACGAGAAG
CGCTGGTTGGACGAGGGCAACGGCCGGGTCTTGCTCTTTCAGTTGAGCGGCCCCAT
GATTTTTGGTGTGGCCAAGGCGATTGCCCGCGAGCACAACGCCATTCAGGAGTGCG
CCGCCATCGTGTTCGACTTGAGCGACGTGCCCCACTTGGGCGTGACTGCTTCCTTGG
CCTTGGAGAACGCCATTGAGGAGGCCGCCGAGAAGGGTCGGGCTGTTTACATCGTG
GGGGCCACCGGGCAGACCAAGCGCCGCTTGGAGAAGTTGCAGGTGTTTCGCTTCGT
GCCTGAGTCCAACTGCTACGACGACCGCTCCGAGGCCCTCAAGGACGCGGTGCTCG
CTTTGGGTCCTCACGAGTCCGAGGATTCCCCTTCCAGCTCCAGCGTCCAGACCACGT
ACTAA.

### Example 2

### Assembling BICA Overexpression Cassette

For the instant example, pNOC-ARS-stacked-NeoR-GFP-aequorin plasmid was obtained from AddGene (Plasmid #101011). Molecular cloning techniques generally known in the art were conducted to assemble the genetic circuits. In brief, codon optimized BICA gene from Synechocystis sp. PCC 6803 was synthesized from IDT as gBlock gene fragment. MluI/SacI flanking restriction sites were added to the BICA gBlock gene fragment to swap the aequorin gene in pNOC-ARS-stacked-NeoR-GFP-aequorin plasmid to attain an N-terminal GFP fused BICA overexpression construct. Synthetic product was cloned in pJET subcloning vector and sequence verified using pJET forward and reverse primers. DNA from error free clones was used for downstream cloning. Both pJET_BICA and pNOC_Stacked_GFP_Aequorin plasmid were digested by MluI/SacI restriction enzymes. Ligation reaction was performed using T4 DNA ligase (New England Biolabs).

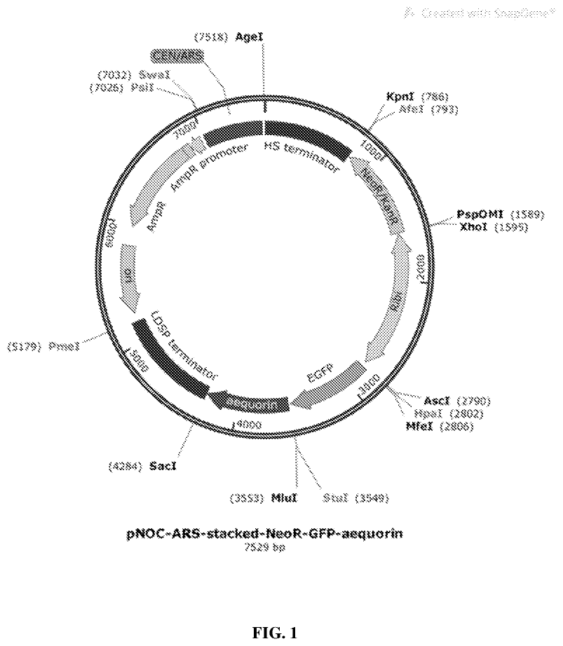

By replacing aequorin, N-terminal GFP fused BICA protein (pNOC_Stacked_GFP_BICA) construct was constructed. The Neomycin resistance gene (NeoR) in the expression plasmid which conferring resistance to the antibiotic G418 was used as a selection marker for Nannochloropsis oceanica transformation. The plasmid is presented in FIG. 1.

### Example 3

### Genetic Transformation

In the instant example, transformation of the exemplary N. oceanica strain CCAP 849/10 was performed by slightly modifying existing electroporation protocols used for other strains of Nannochloropsis (Vieler et al., PLOS Genet. 8, e 1003064, 2012). Exponential growing N. oceanica wild type cells (˜1×10⁷ cells/ml) were aliquoted in 50 ml Falcon tubes and harvested by centrifugation at 3220×g at 4° C. for 15 minutes. Cell pellets were resuspended in 3 ml ice cold 375 mM D-sorbitol, transferred to 2×2 ml Eppendorf tubes, and washed five times in 1.5 ml ice cold 375 mM D-sorbitol. Cell concentration was adjusted to 1-2×10⁶ cells/μl and 100 μl of the cells was aliquoted in 1.5 ml Eppendorf tubes for transformation.

About 3 μg ScaI linearized plasmid DNA and 40 μg of denatured salmon sperm DNA (Invitrogen) were added to the cells and incubated on ice for 10 minutes. Electroporation was performed using a Bio-Rad Gene Pulser Xcell Electroporation System (BIO-RAD), set at 500 Ohms, 25 μF, and 1800 V using a 2 mm cuvette (BIO-RAD). After electroporation, cells were resuspended in 10 ml f/2 medium and allowed to recover for 48 hrs at 25° C. growth chamber in low continuous light regime (˜30 μmol/s/m2). Cells were harvested by centrifugation at 3220×g at 25° C. for 15 minutes, resuspended in 100 μl f/2 medium and plated on f/2 agar plates containing 500 μg/ml Geneticin (G418). Plates were incubated in a growth chamber set at 25° C. until colonies become visible. Two- to three-week-old resistant colonies were transferred to 2 ml of f/2 with 500 μg/ml G418 added for further screening.

### Example 4

### GFP Fluorescence Intensity Analysis

Fluorescence data and cell counts were collected using a BD Accuri C6 flow cytometer (BD Biosciences). Approximately 1 ml of diluted cultures at early exponential growth phase were passed through pre-separation filter (30 μm) to remove debris. Flow cytometry was carried out at a medium flow rate of 35 μL min⁻¹ and a core size of 16 μm. Nannochloropsis cells were counted by gating out cell debris and clumps using the FL3, which measures fluorescence emissions greater than 670 nm. Mean eYFP fluorescence intensities were measured using FL1, which allows the analysis of fluorescence at 533/30 nm.

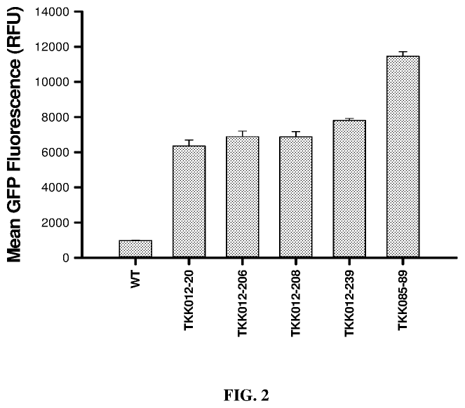

FIG. 2 shows the flow cytometry analysis of cells expressing Green Fluorescent Protein (GFP) versus untransformed controls (WT). Cells with higher mean GFP fluorescence expressed the BicA-GFP fusion protein (i.e., cells denoted with “TKK”) as compared to wild type (WT).

### Example 5

### DIC Dependent Growth Response Analysis

For the assays of the instant example, N. oceanica wild type strain CCAP849/10 and BICA overexpressing transgenic strain, TKK012-206 were analyzed. Cells were initially maintained at mid-exponential growth phase in low light (˜100 μmol photon m⁻² s⁻²) at 25° C. in f/2 media.

For bicarbonate assay, cultures were diluted to 2.5×10⁵ cells/ml in 50 ml f/2 media without bicarbonate and initial OD680 was measured and adjusted to 0.0077. Cells were grown by shaking in continuous light (˜300 μmol photon m⁻² s⁻²) at 25° C. Everyday 5 mM NaHCO₃ was added to the cultures after sampling. Samples (3 ml) were taken every day for OD680 measurement for a period of six days. Data were collected in three biological replicates.

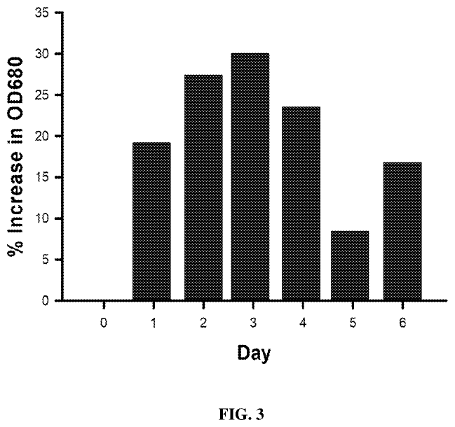

A BicA-GFP expressing strain, TKK012-216, and a control strain were growth with daily additions of 5 mM NaHCO₃. Relative rates of biomass accumulation were measured via the increase in OD680 (optical density at 680 nm of light). The percent increase in daily biomass accumulation of TKK012-216 cells expressing BicA-GFP compared to control cells not expressing BicA-GFP is shown in FIG. 3.

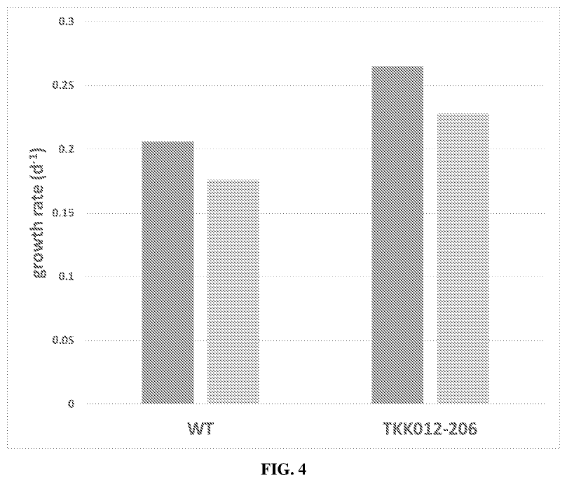

FIG. 4 demonstrates a 29% increase in growth rate of the BicA-GFP expressing strain (TKK012-206) relative to the control strain (WT) with two supplemental additions of 0.2 mM HCO₃⁻ per day. Changes in growth rate are based off of twice daily measurements of biomass using OD680 as a proxy. Each bar represents one biological replicate

### Example 5

### DIC Dependent Photosynthetic Oxygen Evolution Analysis

For the assays of the instant example, Nannochloropsis oceanica wild type strain CCAP849/10 and BICA overexpressing transgenic strain, TKK012-20 were analyzed. Cells were maintained axenically in f/2 liquid medium buffered by 25 mM EPPS, pH 8.2. Cultures were grown in flasks by agitation at 120 rpm. 500 μg/ml G418 was added to the transgenic cultures for selection. Growth temperature was set at 25° C. in continuous lights (˜115 μmole photon m⁻² s⁻¹). Samples (5 ml) were harvested by centrifugation for 10 minutes at 3200×g. Chlorophyll was extracted with 1 ml methanol by boiling at 72° C.

The absorbance of the supernatant at 665 nm was measured by spectrophotometer and chlorophyll concentration was determined following published protocols. Cells equivalent to 25 μg/ml were harvested by centrifugation for 10 minutes at 3200×g and resuspended in fresh ESAW salt media (without added bicarbonate) that was bubbled with nitrogen gas.

Approximately 2 ml of this suspension was used to measure oxygen concentration. Oxygen evolution was measured with a FireSting oxygen meter by illuminating cultures with 319 μmole photon m⁻² s⁻¹ intensity of red light. In each run, before adding the NaHCO₃, the DIC was depleted until no net oxygen evolution is seen. 40 mM NaHCO₃ (final concentration) was then added, and the cuvette was sealed. The maximum oxygen evolution rate was normalized by chlorophyll content.

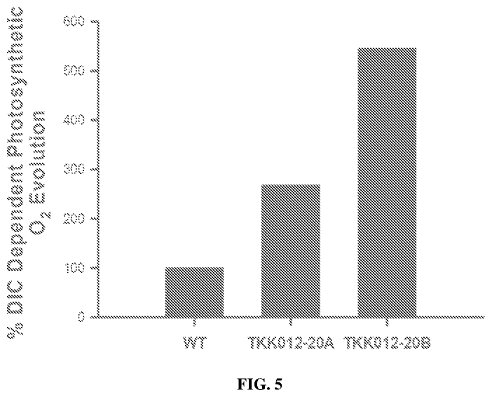

FIG. 5 shows duplicate measurements of photosynthetic activity of an exemplary BicA-GFP expressing strain (TKK012-20) compared to a control strain (WT) that was not transformed with BicA-GFP fusion protein. Oxygen evolution rates are indicative of total photosynthetic activity. Cells were starved for CO₂ and then 40 mM NaHCO₃(DIC) was added to initiate photosynthesis. Photosynthetic rates were followed for 3 minutes.

1. A recombinant polynucleotide comprising a heterologous regulatory element operably linked to a first nucleic acid sequence encoding a cyanobacterial bicarbonate transporter protein.

2. The recombinant polynucleotide of claim 1, wherein the cyanobacterial bicarbonate transporter protein comprises SEQ ID NO: 1.

3. The recombinant polynucleotide of claim 1, wherein the cyanobacterial bicarbonate transporter protein comprises at least 80% sequence identity to the sequence of SEQ ID NO: 1.

4. The recombinant polynucleotide of claim 1, wherein the cyanobacterial bicarbonate transporter protein comprises at least 90% sequence identity to the sequence of SEQ ID NO: 1.

5. The recombinant polynucleotide of claim 1, wherein the cyanobacterial bicarbonate transporter protein comprises at least 95% sequence identity to the sequence of SEQ ID NO: 1.

6. The recombinant polynucleotide of claim 1, wherein the cyanobacterial bicarbonate transporter protein is BicA.

7. The recombinant polynucleotide of claim 1, wherein the heterologous regulatory element is a promoter that functions in eukaryotic cells.

8. The recombinant polynucleotide of claim 1, wherein the promoter is a promoter that functions in eukaryotic algae.

9. The recombinant polynucleotide of claim 1, wherein the first nucleic acid sequence is a native cyanobacterial sequence that has been modified to comprise codons optimized for expression in eukaryotic cells.

10. The recombinant polynucleotide of claim 1, wherein the first nucleic acid sequence is a cyanobacterial bicarbonate transporter gene from Synechocystis that has been modified to comprise codons optimized for expression in eukaryotic cells.

11. The recombinant polynucleotide of claim 1, wherein the first nucleic acid sequence comprises SEQ ID NO: 2.

12. The recombinant polynucleotide of claim 1, wherein the first nucleic acid sequence comprises a sequence having at least 90% sequence identity to SEQ ID NO: 2.

13. The recombinant polynucleotide of claim 1, wherein the first nucleic acid sequence comprises a sequence having at least 95% sequence identity to SEQ ID NO: 2.

14. (canceled)

15. (canceled)

16. (canceled)

17. The recombinant polynucleotide of claim 1, further comprising a second nucleic acid sequence encoding a marker.

18. The recombinant polynucleotide of claim 17, wherein the second nucleic acid sequence is transcriptionally linked to the first nucleic acid sequence and the polynucleotide encodes a fusion peptide comprising the cyanobacterial bicarbonate transporter protein and the marker.

19. The recombinant polynucleotide of claim 17, wherein the marker is a fluorescent marker.

20. (canceled)

21. An expression vector comprising a recombinant polynucleotide of claim 1 and a selectable marker gene.

22. The expression vector of claim 21, wherein the selectable marker gene encodes for antibiotic resistance.

23. The expression vector of claim 21, wherein the selectable marker gene is neomycin (Neo).

24.-37. (canceled)

38. A method of removing carbon from a substance,
said method comprising the step of:
contacting a eukaryotic alga to the substance, wherein the eukaryotic alga comprises an expression vector comprising a recombinant polynucleotide of claim 1 and a selectable marker gene,
wherein the substance comprises carbon, and
wherein the eukaryotic algae removes carbon from the substance.

39.-51. (canceled)


SEQUENCE LISTING (3 sequences)

SEQ ID NO: 1 - protein, 564 aa, Synechocystis thermalis
MQITNKIHFRNLQGDLFGGVTAAVIALPMALAFGIASGAGATAGLWGAVIVGFFAALFGGTPTLISEPTGPMTVVQTAVIASLVAADPDNGLAMAFTVVMMAGLFQIAFGLLKLGKYVTMMPYTVISGFMSGIGIILVILQLAPFLGQASPKGGVIGTLQALPNLVSNVRPVETLLALMTVGIIWFMPSRWKKFAPPQLVALVLGTIISITLFGDLDIRRIGEIQAGLPALQLPVFQADQLQRMLIDAAVLGMLGCIDALLTSVVADSLTRTEHNSNKELVGQGIGNVMSGLFGGLGGAGATMGTVVNIQSGGRTALSGLIRAMVLLVVILGAAKLAATIPLAVLAGIAFKVGVDIIDWGFLKRAHHVSIKGALIMYAVIVLTVLVDLIAAVGIGVFIANILTIDRMSALQSKAVKSISDADDEILLSANEKRWLDEGNGRVLLFQLSGPMIFGVAKAIAREHNAIQECAAIVFDLSDVPHLGVTASLALENAIEEAAEKGRAVYIVGATGQTKRRLEKLQVFRFVPESNCYDDRSEALKDAVLALGPHESEDSPSSSSVQTTY

SEQ ID NO: 2 - DNA, 1695 bp, Synechosystis sp.
  source 1..1695: mol_type = genomic DNA
atgcaaataactaacaaaattcattttaggaacctgcagggggacctttttggcggggttacagcggcggttattgccctgcccatggccttagccttcgggattgcttccggagcaggggctacggccggactctggggggcggtgatcgtagggtttttcgcggccttatttggcggcacccccaccttaatttccgaaccgactgggcccatgacggtggtgcaaacggcggttattgctagtttagtggcggcagatcccgacaatggcttggccatggccttcactgtggtaatgatggcggggttgttccagattgcctttggtctgctcaaattgggcaaatatgtcaccatgatgccctacacagtcatttccggctttatgtccggcattgggattattttggtgattttgcaactggctccctttcttggccaagctagtcccaagggaggggtaatcggcaccctccaggccctccctaacctagtaagcaatgtcaggccggtggaaaccctattggcgctcatgacggtgggcattatttggtttatgccttcccgttggaaaaagtttgctccgccccaattggtggctttagtgttggggacaattatttccatcaccctatttggcgatctggatatccgtcgcattggggaaattcaggccggtttgcccgctctacagctaccagtgtttcaggctgatcaattacagagaatgctgattgatgcggctgttctgggaatgctgggctgtattgatgccctcctgacttcggtggtggctgatagcttgacccgcacagaacataactccaacaaggaattagtcggccagggcatcggcaatgtaatgtccggtttatttggtggcttggggggagctggggccaccatggggacggtggtaaatatccagtccgggggacgcacagctctgtctggcttgatccgggcgatggtgttgctggtggtaattttaggcgcagctaaattggcggctaccattcccctagccgtattggctggtattgcgttcaaagttggggtggacattattgattgggggttcctcaagcgggctcaccatgtctccatcaaaggggccttgattatgtatgccgtcattgtcctgacggtgttggtggatttaattgcggcagtaggtattggtgtatttattgccaatattctcaccattgaccgtatgagtgcgttgcagtccaaagctgtgaaaagtattagcgatgccgacgacgaaattctcctttccgccaatgagaaacgttggctagatgagggcaatggccgggtcttgcttttccaactcagtggcccaatgatttttggggtggccaaggcgatcgccagggaacataatgccattcaagaatgtgccgccattgtttttgatctgagcgatgtgccccatttgggagtaaccgcttccctggccctggaaaatgccattgaagaagcggcggaaaaaggtcgggccgtttacattgtgggggcaacagggcaaaccaagcgacgcttggaaaaattgcaagtgttccgctttgttcctgaaagtaattgctatgacgaccgttctgaagctctcaaggacgctgtcctagctttgggacctcatgaaagtgaggactccccttccagttcttccgtccagaccacatactga

SEQ ID NO: 3 - DNA, 1695 bp, Nannochloropsis oceanica
  source 1..1695: mol_type = genomic DNA
atgcagatcacgaacaagattcacttccgaaacttgcagggtgacttgtttggtggtgtcacggctgccgtcattgccttgcctatggccttggccttcggtattgcgtccggcgctggtgccacggctggtttgtggggggctgtgatcgtcggtttcttcgcggccttgtttggcggcacccctaccttgatttccgagcctaccggccccatgacggtcgtgcagacggcggtgattgcgtccttggtggcggccgaccccgacaacggcttggccatggcctttacggtggtcatgatggcggggttgttccagattgccttcggcttgctcaagttgggcaagtacgtcaccatgatgccctacacggtcatttccggcttcatgtccggcatcgggatcattttggtgatcttgcagctcgcccccttcctcggccaggccagccccaagggcggggtcatcggcaccttgcaggccctccctaacctcgtcagcaacgtccggcccgtcgagacgctcttggcgctcatgacggtgggcattatctggttcatgccttcccgctggaagaagttcgcgcctccccagttggtggccttggtgttggggaccatcatttccatcaccctcttcggcgacctggacatccgccgcattggggagattcaggctggtttgcctgccctccagttgcctgtgtttcaggcggaccagttgcagcgcatgttgattgacgcggctgtgttgggcatgttgggctgcattgacgctctcttgacttccgtggtggctgacagcttgacccgcacggagcacaactccaacaaggagttggtcggccagggcatcggcaacgtgatgtccggcttgttcggcggcttggggggcgctggtgcaaccatggggacggtggtcaacattcagtccggtggccgaacggcattgtccggcttgatccgggcgatggtgttgctggtggtcattttgggcgccgcgaagttggcggccaccatccccctcgccgtgttggccggcatcgcgttcaaggtcggggtggacatcattgactgggggttcctcaagcgggcgcaccacgtctccatcaagggcgccttgattatgtacgctgtcattgtcttgacggtgttggtggacttgattgcggctgtcggcattggtgtgtttattgccaacatcttgaccatcgaccgaatgagcgcgttgcagtccaaggccgtgaagtccatcagcgatgccgatgacgagattctcttgtccgctaacgagaagcgctggttggacgagggcaacggccgggtcttgctctttcagttgagcggccccatgatttttggtgtggccaaggcgattgcccgcgagcacaacgccattcaggagtgcgccgccatcgtgttcgacttgagcgacgtgccccacttgggcgtgactgcttccttggccttggagaacgccattgaggaggccgccgagaagggtcgggctgtttacatcgtgggggccaccgggcagaccaagcgccgcttggagaagttgcaggtgtttcgcttcgtgcctgagtccaactgctacgacgaccgctccgaggccctcaaggacgcggtgctcgctttgggtcctcacgagtccgaggattccccttccagctccagcgtccagaccacgtactaa